(19) United States
(12) Patent Application Publication
(10) Pub. No.: US 2021/0345970 A1
(43) Pub. Date: Nov. 11, 2021

(54) COMPUTER AIDED DIAGNOSTIC SYSTEMS AND METHODS FOR DETECTION OF CANCER

(71) Applicant: UNIVERSITY OF LOUISVILLE RESEARCH FOUNDATION, INC., LOUISVILLE, KY (US)

(72) Inventors: AYMAN S. EL-BAZ, LOUISVILLE, KY (US); AHMED SOLIMAN, LOUISVILLE, KY (US); AHMED SHAFFIE, LOUISVILLE, KY (US); GURUPRASAD A. GIRIDHARAN, LOUISVILLE, KY (US)

(21) Appl. No.: 17/284,582

(22) PCT Filed: Oct. 14, 2019
(86) PCT No.: PCT/US2019/056093

§ 371 Date: Apr. 12, 2021

Related U.S. Application Data
(60) Provisional application No. 62/745,722, filed on Oct. 15, 2018.

(51) Int. Cl. A61B 5/00; A61B 5/107; A61B 5/08; G06N 3/04; G16H 50/20

(52) U.S. Cl. CPC A61B 5/7264; A61B 5/1075; G16H 50/20; G06N 3/04; A61B 5/082

(57) ABSTRACT

A computer-aided diagnostic (CAD) system and method for non-invasive detection of cancer includes receiving and analyzing data from a plurality of sources, using a neural network to generate an initial classification probability from each data source, assigning weights to the initial classification probabilities, and integrating the initial classification probabilities to generate a final classification. The final classification may be a designation of a tissue, such as a pulmonary nodule, as cancerous or noncancerous.

This application claims the benefit of U.S. provisional patent application Ser. No. 62/745,722, filed 15 Oct. 2018, for COMPUTER AIDED DIAGNOSTIC SYSTEMS AND METHODS FOR DETECTION OF CANCER, incorporated herein by reference.

### FIELD OF THE INVENTION

A computer-aided diagnostic (CAD) system and method for non-invasive detection of cancer includes receiving and analyzing data from a plurality of sources, using a neural network to generate an initial classification probability from each data source, assigning weights to the initial classification probabilities, and integrating the initial classification probabilities to generate a final classification. The final classification may be a designation of a tissue, such as a pulmonary nodule, as cancerous or noncancerous.

### BACKGROUND

Lung cancer is a leading cause of morbidity and mortality in the US. Early diagnosis of lung cancer significantly improves the effectiveness of treatment and increases the five-year survival rate from 17.7% to 55.2%. Further, it has been demonstrated that patients with smaller, early stage tumors have a much higher survival rate than patients with larger than T1 tumors.

Early detection of lung cancer (non-small cell, small cell, and carcinoid tumors) currently depends on imaging techniques, primarily computed tomography (CT) scanning. that identify small nodules (stage IA), of which only 6% are cancerous. Discrimination between benign and cancerous lung nodules requires that patients be followed for up to two years to estimate nodule growth rate. This current clinical practice subjects patients to multiple CT scans, resulting in significant diagnostic costs and radiation exposure. Diagnosis may also require bronchoscopy, percutaneous biopsy, or surgical interventions.

Lung cancer diagnosis currently depends upon imaging techniques and the developing technology of breath analysis. The advent of CT scanning has enabled large-scale screening for lung cancer using imaging techniques. The National Lung Screening Trial (2011) detected a high proportion of early cancers (49% stage IA) using CT scans, allowing for intervention with curable intent, which resulted in a 20% reduction in lung cancer mortality. However, among the 24.2% of patients who tested positive for lung cancer, only 6% had a cancerous tumor. The remaining 94% of false positive cases were primarily benign pulmonary nodules that required further investigations including serial CT scanning, positron emission tomography (PET), bronchoscopy, percutaneous biopsy or surgical intervention for the correct diagnosis. Serial CT scanning to observe growth, resolution, or stability is commonly used for sub-centimeter lesions. PET scintigraphy of the lesion may be used to assess the probability of malignancy in nodules larger than 8 mm. PET scans are frequently falsely positive both in cases of solitary pulmonary nodules and in cases with hilar and mediastinal adenopathy. These factors lead to increased clinical suspicion of lung cancer and an obligation to rule out malignancy by surgical intervention. Bronchoscopy and percutaneous biopsy are options for tissue acquisition but the associated costs and risks are significant. In addition, diagnostic yields are highly conditional with respect to tumor size, location, and operator skill. Proceeding directly to surgical resection is appropriate when the probability of malignancy is high and the surgical risk is low. Surgical resection of nonmalignant disease is a clinical failure as a benign nodule would never have harmed the patient. The prohibitive costs associated with repeated radiographic scans and the morbidity due to unnecessary invasive procedures for benign nodules necessitate the development of new diagnostic assays that can detect malignant pulmonary nodules (lung cancer).

Various computational methods exist for classification of lung nodules detected in CT scans. However, despite requiring multiple serial CT scans over a two year period, these methods have a low classification accuracy for early diagnoses of lung cancer because they: 1) do not account for large deformations in lung tissue due to breathing and beating of the native heart; and 2) do not use the 3D shape and appearance of detected nodules in conjunction with estimated nodule growth rate. Importantly, these methods are unsuitable for certain types of lung nodules (e.g. cavities and ground glass nodules), and can be difficult for clinical practitioners to use as they require significant graphic interaction.

Breath analysis of patients is a developing modality for non-invasive detection of cancer originating in the lungs or cancer which has spread to the lungs from a non-lung origination location. Oxidative stress produced by the variable redox environment within cancer is thought to increase the production of various volatile organic compounds (VOCs), which are exhaled in breath. Several approaches include sensor arrays, proton transfer reaction mass spectrometry, selected ion flow tube mass spectrometry, and gas chromatography—mass spectrometry. Breath analysis reports on lung cancer patients indicate a host of associated compounds and profiles; however, the diagnostic usefulness of breath analysis has not been established by these results. The composition of breath ranges from molecular hydrogen to more than 1,000 volatile organic compounds and nonvolatile condensates. Applicant previously developed a breath test and identified 4 specific VOC markers that are indicative of cancer, as described in U.S. Pat. No. 9,638,695, incorporated herein by reference. The concentrations of these VOCs are elevated in cancer patients in comparison to normal controls, healthy smokers, and patients with benign pulmonary disease. The breath test comprises a patient delivering one tidal volume of breath into a non-reactive bag. The breath sample is evacuated through a closed microfluidic chamber, which selectively captures VOCs that are eluted and analyzed by mass spectrometry. This method is quantitative, inexpensive, and reproducible, but the accuracy, sensitivity and specificity of the breath test alone does not exceed 80%, which is lower than thresholds required for reliable diagnosis.

Limitations of existing methods for early detection of lung cancer include the following: (A) Diagnostic specificity, sensitivity, and accuracy based on a single CT is low (80% accuracy). Thus, a single CT scan has limited diagnostic usefulness in early stage lung cancer detection and diagnosis. (B) Breath analysis for lung cancer has approximately 80% accuracy, sensitivity, and specificity, which is lower than the 95% threshold required for reliable diagnosis. Thus, breath analysis alone has limited diagnostic usefulness. (C) Most existing approaches predict the malignant potential of non-calcified nodules based on estimating the growth rate. Absence of growth must be documented over a two-year time period, which entails multiple CT scans with associated costs and radiation exposure. The two-year time frame for diagnosis delays treatment, decreases lung cancer survival rate, and increases treatment costs. (D) Most methods depend solely on CT markers and do not integrate other biomarkers (e.g. VOCs). Thus, these methodologies require up to 2 years to classify the detected lung nodules as benign or malignant. (E) Some imaging methods depend on the Hounsfield Unit values (HU) as the appearance descriptor without taking any spatial interactions into consideration. (F) Most imaging methods that depend on traditional shape features, like curvature, are very sensitive to pre-processing steps, e.g., segmentation.

There is an urgent clinical need for new, non-invasive technologies that will accurately and rapidly diagnose small, malignant lung nodules at early stages, as well as large nodules located away from large-diameter airways that current technologies, such as needle biopsy and bronchoscopy, fail to diagnose.

### SUMMARY

Applicant previously developed a chemical pre-concentrator and a breath test, and identified 4 specific VOC markers that are indicative of cancer, as described in U.S. Pat. Nos. 9,638,695 and 8,663,581, both incorporated herein by reference. In addition, Applicant previously developed a CAD system incorporating shape analysis for diagnosing malignant lung nodules, as described in U.S. Pat. No. 9,230,320, incorporated herein by reference. Disclosed herein is a novel computer-aided system and method for non-invasive detection of cancer which includes receiving and analyzing data from a plurality of sources, using a neural network to generate an initial classification probability from each data source, assigning weights to the initial classification probabilities, and integrating the initial classification probabilities to generate a final classification. Exemplary sources of data include CT scans and breath analysis, as described in Applicant's above-referenced patents, but other biomarkers of cancer may be used as well.

It will be appreciated that the various systems and methods described in this summary section, as well as elsewhere in this application, can be expressed as a large number of different combinations and subcombinations. All such useful, novel, and inventive combinations and subcombinations are contemplated herein, it being recognized that the explicit expression of each of these combinations is unnecessary.

### BRIEF DESCRIPTION OF THE DRAWINGS

A better understanding of the present invention will be had upon reference to the following description in conjunction with the accompanying drawings.

FIG. 1 is a schematic diagram depicting the integration of imaging data and clinical data using deep-learning based techniques to generate a diagnosis.

FIG. 2 is a depiction of Spherical Harmonics (SHs) used to approximate the 3D shape for malignant and benign nodules.

FIG. 3A depicts a benign nodule (top row) and malignant nodule (bottom row) via 2D visualization of HU values (A) and 3D visualization of HU values (B) of axial cross sections of the nodules.

FIG. 3B depicts benign (top row) and malignant (bottom row) nodules via CT scan (A), 3D visualization of HU values (B), and calculated Gibbs energy values which display as higher energy (brighter) for benign and lower energy (darker) for malignant nodules (C).

FIG. 4 is a schematic illustration of coating 2-(aminooxy)-N,N,N-trimethylethanammonium (ATM) and chemical reactions with carbonyl compounds.

FIG. 5 is a diagram depicting a system for capture of carbonyl VOCs in exhaled breath.

FIG. 6 is a Fourier transform ion cyclotron resonance mass spectroscopy (FT-ICR-MS) spectra of breath samples processed through ATM-coated microreactors for (a—top) lung cancer patient, (b—middle) a healthy current smoker, (c—bottom) a healthy non-smoker.

FIG. 7A is a schematic diagram of the appearance diagnostic network of FIG. 1.

FIG. 7B is a schematic diagram of shape diagnostic network of FIG. 1.

FIG. 7C is a schematic diagram of the breath diagnostic network of FIG. 1.

FIG. 7D is a schematic diagram of the final diagnosis neural network of FIG. 1.

### DETAILED DESCRIPTION OF THE PREFERRED EMBODIMENTS

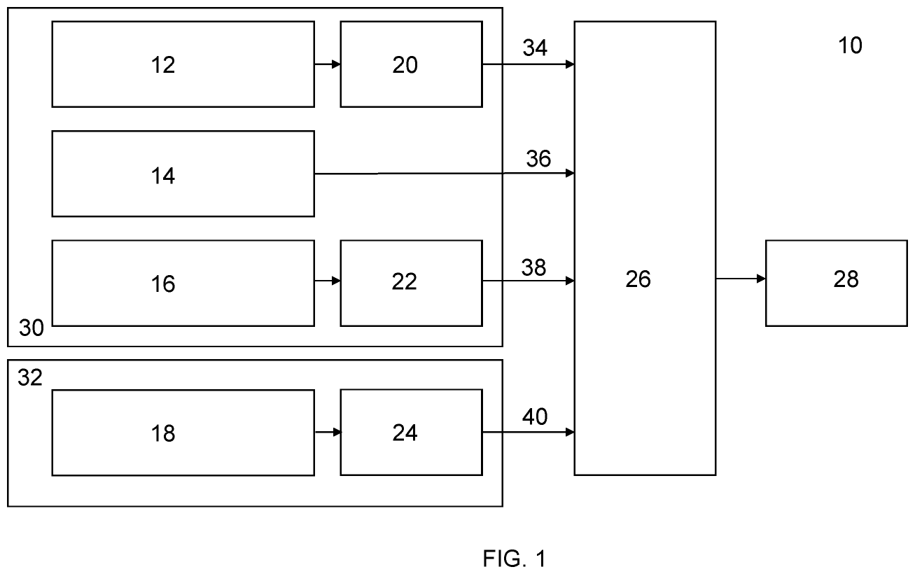

The present invention comprises CAD systems and methods, summarized in FIG. 1, that integrate clinical data, such as biomarker data from a patent's exhaled breath, with clinical data, such as image-based CT markers, to provide an accurate and rapid diagnosis of small lung nodules or other tissue of interest. Combining both breath biomarker and imaging data significantly improves the accuracy (>95%), sensitivity, specificity, and speed (several days instead of two years) of early-stage lung cancer diagnosis, and does so non-invasively using only a single CT scan and a breath test.

As an overview, the method 10 comprises receiving a plurality of measureable indicators of the presence or absence of a cancer disease state in a subject. In some embodiments, these indicators are imaged-based biomarkers derived from CT scans, such as, for example, appearance indicators 12 based on the three dimensional appearance of lung nodules stated in terms of Gibbs energy, size indicators 14 of lung nodules, and shape indicators 16 based on the shape of lung nodules in terms of the number of spherical harmonics (SHs) required to approximate the complex 3D shape of the nodules. In some embodiments, these indicators further include clinical-based biomarkers, such as, for example, indicators based on the volatile organic compound content of the patient's exhaled breath 18. With respect to breath analysis, the technology as described in U.S. Pat. Nos. 9,638,695 and 8,663,581 enables chemoselective capture (>95% capture rate) of carbonyl compounds in exhaled breath and concentrates carbonyl compounds by over 6,600-fold for quantification using mass spectrometry.

Machine learning techniques are used to generate an initial classification probability for at least one, or a plurality of the indicators. In the depicted embodiment, an appearance diagnostic network 20 is a neural network including three hidden layers and a softmax layer which is used to generate an initial classification 34 of benign or malignant for the lung nodule based on the appearance indicators 12. A shape diagnostic network 22 is a neural network including three hidden layers and a softmax layer which is used to generate an initial classification 38 of benign or malignant for the lung nodule based on the shape indicators 16. A breath diagnostic network 24 is a neural network including three hidden layers and a softmax layer which is used to generate an initial classification 40 of benign (noncancerous) or malignant (cancerous) for the lung nodule based on the breath indicators 18. An initial classification 36 of benign or malignant for the lung nodule may be generated directly based on the size indicators 14 without using parametric methods. The individual initial classifications 34, 36, 38, 40 generated based on indicators 12, 14, 16, 18 are each input into a final diagnosis neural network 26 which assigns a weight to each initial classification, then generates a final classification 28 of the lung nodule as benign or malignant (i.e., determining the presence or absence of a cancer disease state) by integrating the initial classification probabilities based on their respective weights. This method combines image-based biomarker data 30 and clinical-based biomarker data 32 to generate an early-stage classification of a nodule as benign (noncancerous) or malignant (cancerous). Each of these steps are explained in further detail below.

Patient Sample. In a pilot clinical study (n=47 patients), CT and breath analysis data were both collected on the same day from 47 patients (Table I). One liter of mixed tidal and alveolar breath sample was collected into a non-reactive Tedlar™ bag (Sigma Aldrich, St Louis, Mo.) from a single exhalation from each participant. The CT data was collected from the same 47 patients with a slice thickness of 2.5 mm reconstructed every 1.5 mm, KV 140, MA 100, and F.O.V 36 cm. The ground truth for nodule detection and segmentation was obtained by the union of the masks of nodules that were manually segmented by three radiologists. Patient selection was blinded but included patients with both benign and malignant small lung nodules (4 to 20 mm) and large nodules (>20 mm). The patient diagnostic conclusions were blinded from the data analysis team for lung cancer diagnosis using both breath test and CT markers. The patients were either biopsied for diagnostic conclusion or followed for up to two years until a final lung cancer diagnosis could be determined based on current clinical approaches (serial CT scans and/or biopsy/bronchoscopy). The accuracy, sensitivity, and specificity of the proposed CAD system were determined based on the final lung cancer diagnosis using conventional clinical methods (ground truth).

TABLE I
Demographics and nodule size of patients (n = 47 patients).
D = nodule diameter.

| | Subject | Male | Female | Nodule Size |
|---|---|---|---|---|
| Malignant | 20 | 3 | 17 | 4 mm ≤ D ≤ 20 mm |
| | 17 | 9 | 8 | 20 mm ≤ D ≤ 60 mm |
| Benign | 5 | 1 | 4 | 20 mm ≤ D ≤ 60 mm |
| | 5 | 5 | 0 | 20 mm ≤ D ≤ 34 mm |

To mitigate the limited sample size of patients with both breath and imaging data, retrospective CT scans from 467 patients with 727 nodules (benign=413, malignant=314) from the Lung Image Database Consortium (LIDC) database were used to validate the classification methodology. The nodules were detected, delineated, and diagnosed by four radiologists, where each of them assigned a malignancy score on scale of 1 to 5 (1 represents benign and 5 represents malignant). The 727 nodules used for validation were those which had a high degree of confidence and agreement between the four radiologists. Specifically, only nodules that received an average score of 3.5 or greater (deemed malignant) and nodules with an average score of 1.5 or lower (deemed benign), were included in this study. For breath analysis, samples from 504 patients were collected (benign=252, malignant=252) and analyzed. The malignant nodules were confirmed by pathological diagnosis and the benign ones were confirmed by tissue diagnosis or repeated CT scans with no discernable change or decrease in size for ≥2 years.

Imaging Markers from CT Data. The disclosed approach accurately delineates lung nodules from surrounding lung tissues (segmentation) to measure the size, and analyze the shape and appearance of detected nodules. Imaging markers based on nodule size, shape, and appearance analyses are used as discriminatory features to distinguish between malignant and benign lung cancer nodules at an early stage. The following three parameters are calculated from CT imaging: (1) size (i.e., diameter, representative of volume) of lung nodules, (2) shape of lung nodules in terms of the number of spherical harmonics (SHs) required to approximate the complex 3D shape of the nodules, and (3) 3D appearance of lung nodules based on the Gibb's energy. To calculate CT markers that describe the shape, appearance, and size/volume of the detected lung nodules, the initial nodule location is determined, followed by lung nodule segmentation, followed by analysis of the size, shape, and appearance of the segmented nodule. The detected lung nodules will be initially classified as either malignant or benign based on the calculated CT markers.

Segmentation of Lung Nodules. To separate each pulmonary nodule from its background in a chest CT image, two adaptive probabilistic models of the visual appearance of small 2D and large 3D pulmonary nodules are used as disclosed in U.S. Pat. No. 9,230,320 to control the evolution of a deformable boundary. The prior appearance will be modeled with a translation and rotation-invariant Markov Gibbs Random Field (MGRF) with pairwise interaction of voxel intensities. The MGRF is identified analytically from a set of training nodules and the visual appearance of the nodules will be represented with a mixed marginal probability distribution of voxel intensities in chest images (modeled with the Linear Combination of Discrete Gaussians model). This approach accurately separates the pulmonary nodules from attached vessels, and has a low error (0.98%) compared to radiologist's “ground truth”.

Size Analysis. Larger sized nodules have a higher probability of being malignant. Classification based on nodule size, while straightforward, is not suitable for nodule classification for early stage lung cancer where nodule sizes are usually between 4 mm to 30 mm. A k-nearest neighbor (K-NN) classifier was fed with the nodules' size data and was used to give an initial malignancy probability for each pulmonary nodule.

3D Shape Analysis. Malignant nodules grow faster than benign nodules and thus have a more complex shape and surface. Surface shape complexity was quantified using spherical harmonic (SH) decomposition. Malignant nodules with complex surfaces require more SHs than the smoother benign nodules, enabling classification between malignant and benign nodules. Briefly, a spectral SH analysis was used to model the pulmonary nodules, by considering its surface as a linear combination of particular basis functions. After the triangulated 3D mesh is built, it is mapped to the unit sphere for the SH decomposition. A new mapping approach, the Attraction-Repulsion Algorithm, was developed to ensure that: (i) the distance from the center of the nodule to any node as unity, and (ii) each node is equidistant to all its neighbors.

Let I refer to the number of mesh nodes, α the cycle iterator, and C_{α,i} the coordinates of node i at cycle number α. Let J refer to the number of neighbors for the mesh node and d_{α,ij} denote the Euclidean distance between i and j at cycle number α, where j=1, . . . , J. Let d_{α,ji}=C_{α,j}−C_{α,i} denote the displacement between the nodes j and i at cycle number α. Let C_{A,1}, C_{A,2}, C_{R} be the constants controlling the displacement for each surface node. The attraction step adjusts the location for each node Cᵢ to be centered with respect to its neighbors and is given by:

$${{C}^{′}}_{α , i} = {C}_{α , i} + {C}_{A , 1} {\sum }_{j = 1 ; j \neq i}^{J} {d}_{α , ji} {d}_{α , ji}^{2} + {C}_{A , 1} \frac{{d}_{α , ji}}{{d}_{α , ji}}$$

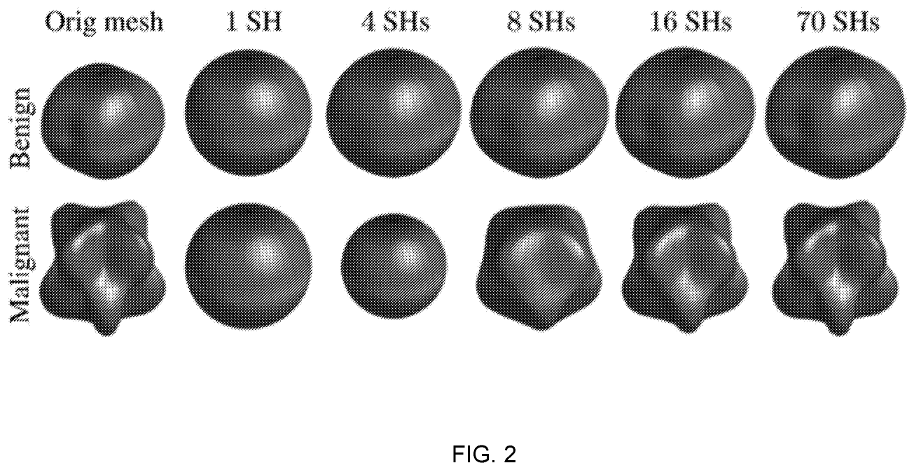

The nearer nodes are pushed further from each other, while C_{A,2} keeps the nodes from collapsing. Thus, the entire mesh is inflated in the repulsion step by pushing every node outward to preserve the equidistant condition after their last back-projection onto the unit sphere along the rays from the sphere's centroid. In the repulsion step, every node is pushed outward to maintain the equidistant condition after their last back-projection onto the unit sphere along the rays from the sphere's centroid. To avoid overlap or crossing over of nodes during shifting, the location for each Ci is updated after the back-projection as:

$${C}_{α + 1 , i}^{o} = {{C}^{′}}_{α , i} + \frac{{C}_{R}}{2 I} {\sum }_{j = 1 ; j \neq i}^{I} ( \frac{{d}_{α , ji}}{{\vert {d}_{α , ji} \vert}^{2}} )$$

where CR is the repulsion constant. After the mapping process, the nodule surface was approximated by a linear combination of SHs. Lower-order harmonics will be adequate to approximate a more uniform shape (benign nodules), compared to higher-order harmonics for more complex shapes (malignant nodules), as shown in FIG. 2. In other words, the number of SHs required to approximate nodule shape can be used for nodule classification. The SHs coefficients from up to 70 harmonics were subsequently used to reconstruct the original pulmonary nodule.

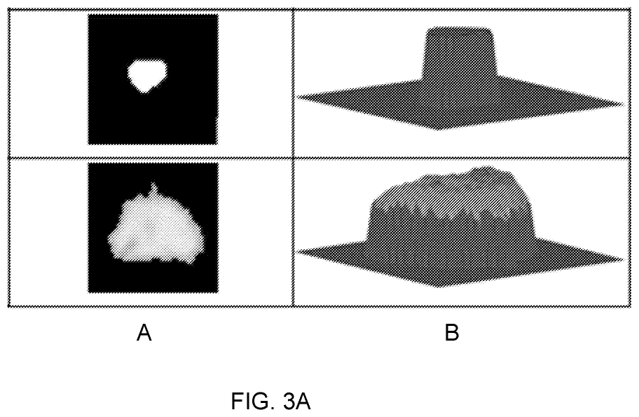

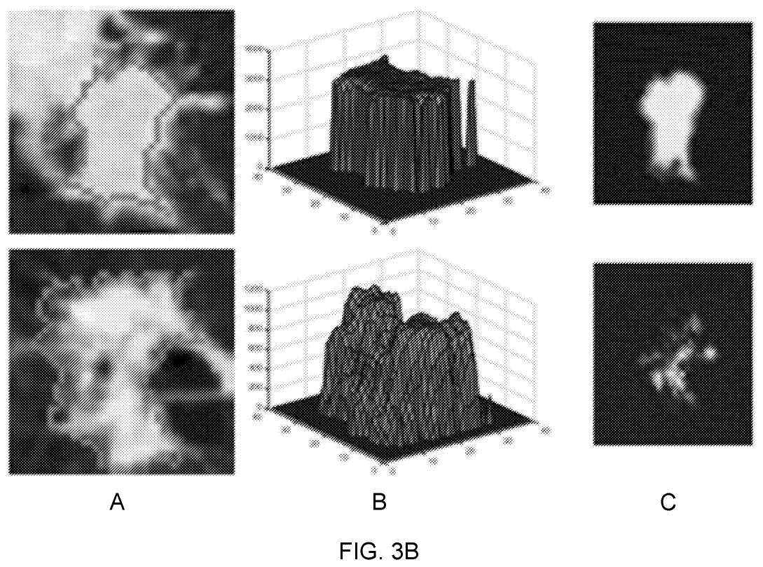

3D Appearance Analysis. Malignant nodules, due to their high growth rate, have a non-uniform density (spatial non-homogeneity) compared to benign nodules, which is reflected as varying Hounsfield units (HU) in the CT scan (FIG. 3A). HU is a unit of measure that represents the different levels of tissue density as visualized in the CT images. The Appearance analysis is modeled for the 3D nodule volumes in a way that the differences between the HU of a voxel and its 7 nearest neighbors is represented as Gibb's energy using a 7^{th} order Markov-Gibbs random field(MGRF). Grayscale patterns of the nodules were considered as samples of a trainable translation- and contrast-offset-invariant 7^{th} order MGRF. The MGRF model uses a general-case exponential distribution to relate the image texture probability, g=(g(r):r ∈ [symbol] ), with voxel-wise HU, g(r) to the Gibbs energy, E₇(g), using the following equation:

$${P}_{z} ( g ) = \frac{1}{Z} ψ ( g ) exp ( - {E}_{7} ( g ) )$$

where Z is the normalization factor.

The signal interactions between each voxel and its adjacent seven neighbors, were quantified as simultaneous partial relations between the voxel-wise signals within a fixed distance, ρ, from that voxel. These quantified values are used to describe the visual appearance of the given nodules. A set of known training images, g°, were used in learning the Gibbs potentials, v_{7:p}(g(r′):r′∈R(r)), of translation-invariant 7-voxel subsets. Consequently, the Gibbs energy E₇(g), is computed using the maximum likelihood estimates (MLE) that generalize the similar analytical approximations of the potentials as in the generic 2^{nd}-order MGRF in

$${υ}_{7 : ρ} ( β ) = \frac{{F}_{7 : ρ : core} ( β ) - {F}_{7 : ρ} ( β : {g}^{o} )}{{F}_{7 : ρ : core} ( β ) ( 1 - {F}_{7 : ρ : core} ( β ) )} ; β ∈ {𝔹}_{7}$$

where β is a numerically coded relation between neighboring configuration; [symbol] ₇ represents the set of all possible ordinal 7-signal relations; F_{7:p}(g°) is the marginal probability of the code β;β∈ [symbol] ₇, over all the possible configurations for the seven neighbors with the center-to-voxel distance ρ in g°, and F_{7:ρ:core}(β) is the like probability for the core distribution. The final computed Gibbs energy indicates whether the nodule is malignant or benign, as shown in FIG. 3B. These results demonstrate the ability of using Gibb's energy as a marker for cancer diagnosis. The voxel-wise Gibbs energies for the three similar 7^{th} order MGRFs are used to quantify the nodule appearances.

[symbol]

(r=(x,y,z))={(x,y,z);(x±β,y,z),(x,y±ρ,z),(x,y,z±ρ)}

The potentials and distances, p, between the central voxels and its neighbors are learned from the training image, g°. The output features from the MGRF appearance model is a vector of size 1000 describing the histogram bins of the Gibbs energy for each nodule.

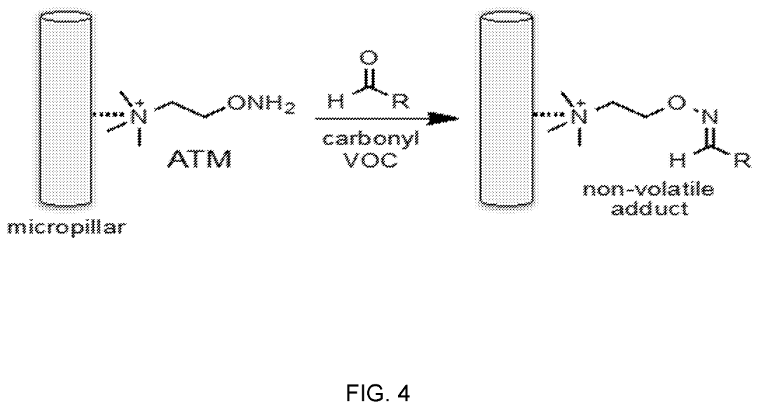

Breath Test Microchip Fabrication and Technology. As described in U.S. Pat. Nos. 9,638,695 and 8,663,581, the breath test technology consists of a silicon microreactor chip, fabricated using microelectromechanical systems (MEMS) fabrication technologies, that concentrates carbonyl compounds from a single 1-liter breath sample. The microreactor chip consists of thousands of micropillars in a microfluidic channel to uniformly distribute a collected breath sample through the channel. The microfluidic channel and the micropillar array were created by deep reactive ion etching (DRIE). The microchips then were sealed by anodic bonding of a glass wafer onto the silicon wafer. The micropillars have a high-aspect-ratio with dimensions of 50 μm in diameter and 350 μm in height. The device structure enables uniform distribution of breath to maximize the reaction of VOCs in the breath with ATM iodide coated on the micropillars as shown in FIG. 4. ATM chemoselectively reacts to trap carbonyl compounds in exhaled breath by means of oximation reactions. The process of concentrating the carbonyl biomarkers on the micropillar surfaces, while allowing the rest of the breath VOCs to pass through the microreactor has been verified using clinical breath samples. Carbonyl VOC capture efficiencies>95% with concentration of carbonyl compounds by over 6,600 fold have been demonstrated. The microreactor chips thus can be used for quantitative analysis of carbonyl VOCs in breath. We have demonstrated carbonyl VOCs to be elevated in the breath of lung cancer patients. The increased carbonyl VOC concentrations have been linked to the high oxidative stress in cancer causing lipid peroxidation.

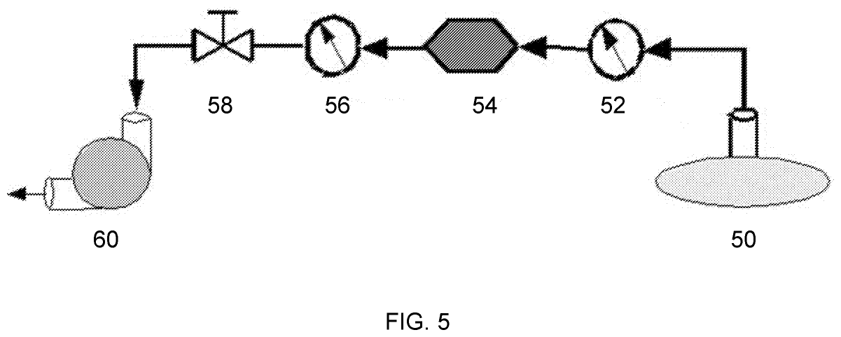

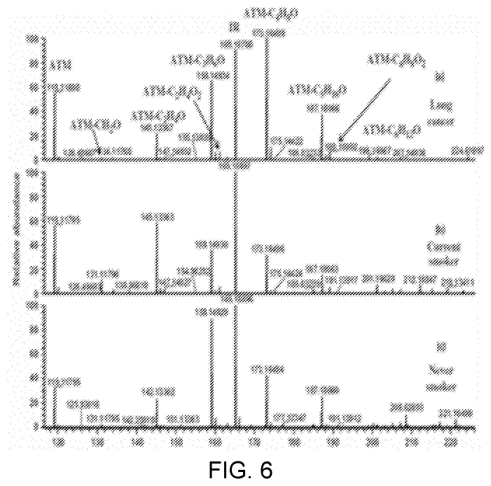

Breath Analysis and Significance of the Pilot Data. Referring now to FIG. 5, the exhaled breath samples were collected from patients in sample bags 50, such as the 1-L sample bags offered by Tedlar™. Then, the gaseous breath samples were drawn sequentially from the bags 50 through a flowmeter 52, preconcentrator microreactor chip 54, pressure gauge 56, and valve 58, by applying a vacuum with pump 60. The arrows in FIG. 5 show the direction of airflow. After this process, the ATM adducts in the microreactor chip 54 were eluted with 100 μL of methanol from a pressurized vial. Ninety-nine percent of ATM adducts were recovered and the eluted solution was analyzed by mass spectrometry. The concentrations of some carbonyl VOCs, specifically 2-butanone (C₄H₈O₂), 3-hydroxy-2-butanone (C₄H₈O₂), 2-hydroxyacetaldehyde (C₂H₄O₂), and 4-hydroxy-2-hexenal (C₆H₁₀O₂), in the exhaled breath of lung cancer patients are significantly higher than in the breath of either healthy controls, healthy smokers, or patients with benign pulmonary nodules (FIG. 6).

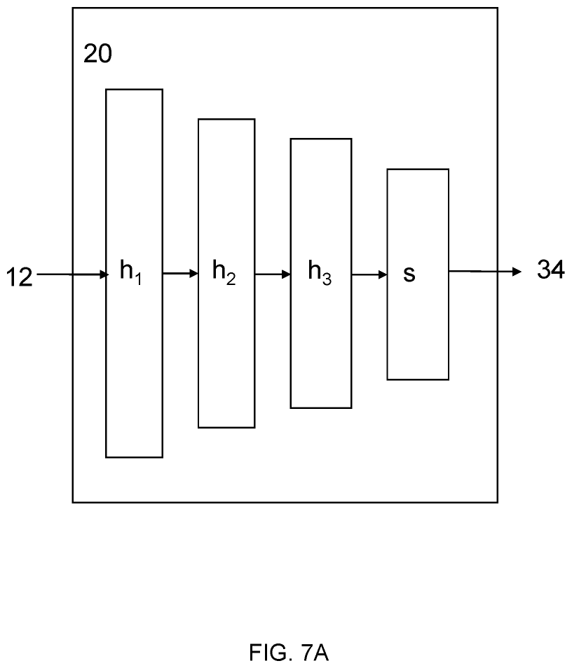

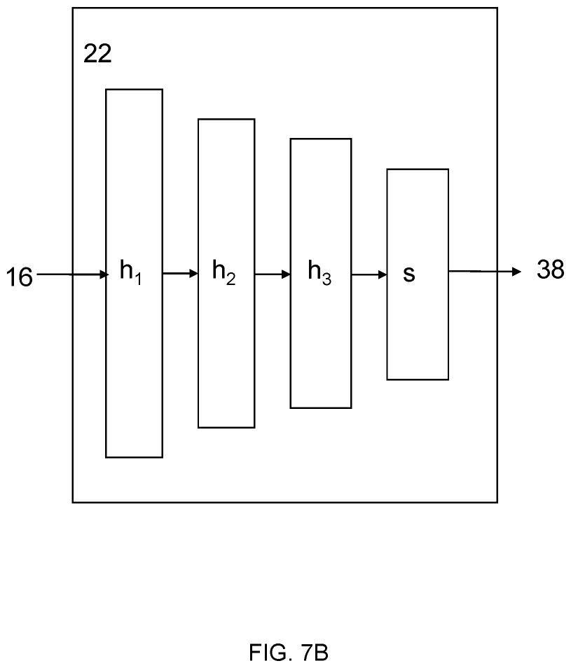

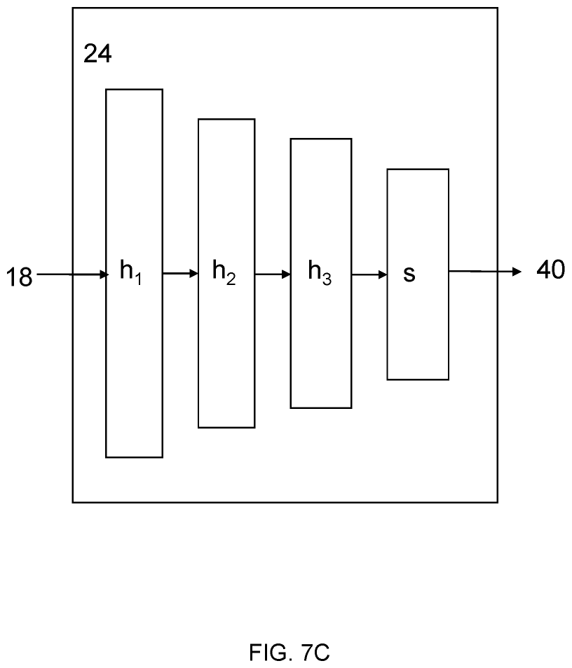

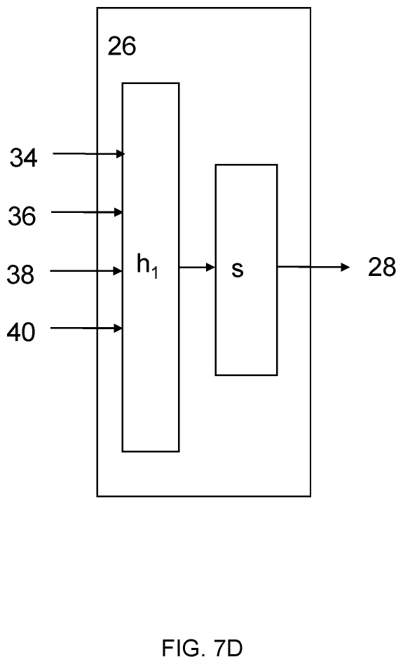

Nodule Classification Using Autoencoders. The CAD system 10 uses a hierarchical or “deep” neural network with a two-stage structure of stacked autoencoder (AE). In the first stage, three autoencoder-based neural network classifiers (appearance diagnostic network 20, shape diagnostic network 22, and breath diagnostic network 24, as shown in FIGS. 7A, 7B, and 7C), are employed to provide initial classification probabilities 32, 34, 36. For each of FIGS. 7A, 7B, and 7C, h₁: 1st hidden layer, having 50 hidden nodes, h₂: 2nd hidden layer, having 30 hidden nodes, h₃: 3rd hidden layer, having 10 hidden nodes, s: softmax layer for generating initial classification probabilities. For appearance diagnostic network 20, the input appearance indicators 12 include, in some embodiments, 200 histogram bins for Gibb's energy. For shape diagnostic network 22, the input shape indicators 16 include, in some embodiments, SHs coefficients from up to 70 harmonics. For breath diagnostic network 24, the input breath indicators 18 include, in some embodiments, detected concentrations of up to 27 exhaled VOCs.

The initial classification probabilities 32, 34, 36, together with the probabilities of the k-NN classifier for size were then input into the final diagnosis neural network 26 (second stage autoencoder) to create the final classification, as summarized in FIG. 1 and shown in greater detail in FIG. 7D. Autoencoder was utilized to decrease the dimensionality of the features with three-layered neural networks to identify the most discriminating features by unsupervised pre-training algorithm, although in other embodiments, other dimensionality reducers may be used (e.g., principal component analysis, linear discriminant analysis, generalized discriminant analysis, etc.). The three hidden layers reduced the hidden shape features from 70 (corresponding to 70 SHs) to 10 as shown in FIG. 7C, hidden appearance features from 200 (corresponding to 200 histogram bins for Gibb's energy) to 100, as shown in FIG. 7A, and 27 hidden breath features (corresponding to 27 VOCs) to 10, as shown in FIG. 7C.

After the AE layers, a softmax layer was used to boost the diagnosis accuracy by limiting the overall loss of the labeled data during the training. Briefly, for each AE, let W={Wⱼ^{e}; Wᵢ^{d}:j=1, . . . , s;i=1, . . . , n} refer to a set of weights column vectors for encoding, E, and decoding, D, layers, and let T denote vector transposition. The AE alters the n-dimensional column vector u=[u₁, ,uₙ]^{T} into an s-dimensional column vector h=[h₁, , hₛ]^{T} of level activators such that s<n by nonlinear uniform transformation of s weighted linear combinations of input as hⱼ=σ((Wⱼ^{e})^{T}u), where σ(.) is a sigmoid function with values from [0,1]

$$σ ( t ) = \frac{1}{1 + {e}^{- t}}$$

The softmax layer calculates the classification probability through the following equation:

$$p ( c ; {W}_{o : c} ) = \frac{{e}^{( {W}_{o : c}^{T} {h}^{3} )}}{{e}^{( {\sum }_{1}^{c} {W}_{o : c}^{T} {h}^{3} )}}$$

where C=1,2; denote the number of the class; W_{o:c}: is the class c weighting vector; h³: are the output features from the last hidden layer, (the third one), of the AE. In the last stage, the output probabilities of the shape, appearance and breath analysis networks, were combined together with the probabilities of the k-NN classifier, and input into a softmax layer to estimate the fused classification decision. A leave-one-subject-out (LOSO) cross validation was used to classify the nodules for the 47 patients with both breath and CT data.

Data from the larger patient cohort (467 patients) with 727 samples (413 benign and 314 malignant) were used to test methodology and classification accuracy of each of the imaging markers. Similarly, the breath analysis data from 504 patients were used to test the methodology and classification accuracy of using breath markers. Due to the large patient cohort, 75% of these CT and breath data was used for training the AE network and 25% data was used for validation.

Results. The classification accuracy, sensitivity, and specificity for each different feature combination for the 47 patients is shown in Table II. Nodule size had the least accuracy and sensitivity while shape and appearance features had the highest accuracy and sensitivity. For the patients for whom both breath and CT data were collected, the integration of all CT and breath markers using the CAD system resulted in accuracy, sensitivity, specificity, and AUC above 97%.

TABLE II
Accuracy, sensitivity, and specificity of
diagnosis (malignant or benign) by feature group.

| | Evaluation Metrics | | |
|---|---|---|---|
| Features | Accuracy | Sensitivity | Specificity |
| Size | 61.19% | 29.73% | 100.00% |
| Shape | 89.55% | 89.19% | 90.00% |
| Appearance | 86.57% | 91.86% | 80.00% |
| Breath Analysis | 75.99% | 71.43% | 80.56% |
| Shape + Size | 91.04% | 89.19% | 93.33% |
| Appearance + Size | 89.55% | 91.89% | 86.67% |
| Shape + Appearance | 91.04% | 94.59% | 86.67% |
| Shape + Breath | 89.55% | 89.19% | 90.00% |
| Appearance + Breath | 88.06% | 91.89% | 83.33% |
| Size + Breath | 79.10% | 72.97% | 86.67% |
| Shape + Size + Breath | 92.54% | 91.89% | 93.33% |
| Shape + Appearance + Breath | 92.65% | 94.74% | 90.00% |
| Appearance + Size + Breath | 92.54% | 94.59% | 90.00% |
| Shape + Appearance + Size | 94.03% | 91.89% | 96.67% |
| All 4 Combined Features | 97.87% | 97.30% | 100.00% |

In the larger patient cohort (breath=504 patients, CT imaging=467 patients), the accuracy, sensitivity, and specificity of size (79.84%, 75.63%, and 83.59%), shape (89.91%, 96.77%, and 84.80%), appearance (89.91%, 93.55%, and 87.20%), and breath (80.95%, 79.69%, and 82.26%) markers were similar to the values obtained with the CAD system for individual markers with the smaller patient cohort (Table II).

Use of the autoencoder allowed for reduction in the dimensionality of the large data set, the ability to extract the most distinguishing features from the data set, and assign different weights to the probabilities generated by each classifier (i.e., appearance, shape, breath analysis, and size) to increase accuracy of the system. The classification accuracy, sensitivity, specificity, and area under the curve (AUC) for each individual marker for the 47 patients is shown in Table II. Nodule size had the least accuracy and sensitivity while shape and appearance features had the highest accuracy and sensitivity. For the patients for whom both breath and CT data were collected, the integration of all CT and breath markers using the CAD system resulted in accuracy, sensitivity, specificity, and AUC above 97%. The stacked AE with softmax combining shape, size, appearance, and breath data had the highest classification accuracy, sensitivity, and specificity amongst all tested classifiers. Table III shows performance metrics for each part of the framework and the fused framework after the combination process using the LI DC dataset for validation.

TABLE III
Accuracy, sensitivity, and specificity of diagnosis (malignant or benign) by
CT imaging modality and in combination using the LIDC dataset.

| | Evaluation Metrics | | |
|---|---|---|---|
| Features | Sensitivity | Specificity | Accuracy |
| Size | 75.63% | 83.59% | 79.84% |
| Shape | 96.77% | 84.80% | 89.91% |
| Appearance | 93.55% | 87.20% | 89.91% |
| Combined Features | 93.55% | 91.20% | 92.20% |

For comparison, in literature, the accuracy, sensitivity, and specificity of diagnosis using various features from CT scans are ˜85% to 90%. As such, the disclosed system combining imaging-based diagnoses with breath analysis-based diagnoses provides significantly improved results with accuracy, sensitivity, and specificity of diagnosis in excess of 97% using only a single CT scan and single breath test.

The CAD system framework is robust to loss of an individual marker, and is capable of integrating additional biomarker data (eg. blood, saliva, urine etc.) to further improve accuracy. The CAD system currently considers 1098 features (1000 appearance features, 70 shape features, size, and 27 VOCs) for nodule classification. A three layered AE network is used to reduce it to 121 features (100 appearance features, 10 shape features, size, and 10 VOCs) that provided the highest discrimination, to minimize computational cost and enable rapid classification. However, greater or lesser reduction of features is also contemplated.

Training the Breath Diagnostic Network. In some embodiments, breath diagnostic network 24 is a deep-learning-based, autoencoder network for assigning an initial classification of cancer or non-cancer based on breath biomarkers. Microreactor chips 54 are used to analyze exhaled breath from healthy controls, patients with lung cancer and patients with benign pulmonary nodules. The breath analysis data is used to train the breath diagnostic network 24 based on elevated concentrations of a panel of carbonyl-containing volatile organic compounds in breath samples of lung cancer patients. The training data was taken from a sample of 200 patients (50 healthy controls and 75 patients with diagnosed lung cancer and 75 patients with benign pulmonary nodules) balanced based on age group, gender, race and smoking status, with matching factors age (+/−5 years), race and gender. The healthy controls had no known inflammatory and malignant disease. Patients with benign pulmonary nodules include COPD, granuloma, and pneumonia. Both the control and patient groups include new referrals for evaluation of pulmonary nodules detected by CT scan and established patients who have already been identified and are undergoing repeat CT scans to follow their pulmonary nodules and not had a biopsy. Once enrolled in the validation study, the patients were followed every 4 months until one of the following conditions are met: 1) confirmed pulmonary malignancy via tissue diagnosis; 2) pulmonary parenchymal abnormality proven to be benign by tissue diagnosis; or 3) pulmonary parenchymal abnormality demonstrated to be stable via serial radiologic exams for a total of 2 years.

The breath diagnostic network 24 was trained based on elevated carbonyl biomarkers in exhaled breath of lung cancer patients in comparison with that in exhaled breath of healthy controls and patients with benign pulmonary nodules. Earlier work identified four carbonyl-containing VOCs as biomarkers in exhaled breath for detection of early stage lung cancer. Here, the autoencoder breath diagnostic network 24 was trained on twenty seven detected carbonyl compounds with measured concentrations. Testing data indicates that using a greater number of carbonyl compounds in exhaled breath can increase the combined diagnostic accuracy, sensitivity and specificity.

As shown in Table IV, a plurality of volatile organic compounds have been individually tested to determine the impact of each one of them separately using autoencoder and softmax classifier. The size of the data used in this experiment is 504 samples (252 benign and 252 malignant). 75% of the data was used for training and 25% for testing.

TABLE IV
Analysis of volatile organic compounds as biomarkers for cancer.

| VOC | Sensitivity | Specificity | Accuracy |
|---|---|---|---|
| C₄H₈O | 77.59 | 73.53 | 75.4 |
| C₅H₁₀O | 66.13 | 81.25 | 73.81 |
| C₄H₈O₂ | 59.7 | 88.14 | 73.02 |
| C₂H₄O₂ | 56.67 | 78.79 | 68.25 |
| C₁₃H₂₂O | 52.38 | 76.19 | 64.29 |
| C₆H₁₂O | 41.18 | 89.66 | 63.49 |
| C₆H₁₂O₂ | 29.69 | 91.94 | 60.32 |
| C₇H₁₄O | 34.85 | 85 | 58.73 |
| C₂H₄O | 46.88 | 69.35 | 57.94 |
| C₁₃H₂₆O | 59.09 | 53.33 | 56.35 |
| C₇H₆O | 19.35 | 89.06 | 54.76 |
| C₅H₈O | 62.32 | 43.86 | 53.97 |
| C₉H₁₆O₂ | 100 | 6.35 | 53.17 |
| C₈H₁₆O | 1.59 | 100 | 50.79 |
| C₄H₆O₂ | 78.79 | 16.67 | 49.21 |
| C₇H₁₁O | 98.41 | 0 | 49.21 |
| C₄H₄O₂ | 100 | 0 | 48.41 |
| C₃H₆O | 100 | 0 | 47.62 |
| C₄H₆O | 95.08 | 1.54 | 46.83 |
| CH₂O | 13.04 | 85.96 | 46.03 |
| C₉H₁₈O | 15.94 | 82.46 | 46.03 |
| C₁₅H₁₀O | 2.99 | 94.92 | 46.03 |
| C₁₀H₂₀O | 35.48 | 54.69 | 45.24 |
| C₁₁H₂₂O | 100 | 0 | 45.24 |
| C₁₂H₂₄O | 100 | 0 | 45.24 |
| C₃H₄₀ | 2.86 | 96.43 | 44.44 |
| C₃H₄O₂ | 100 | 0 | 43.65 |

Combinations of VOCs. To determine the efficacy of combinations of VOCs as biomarkers for cancer, the VOC with the highest accuracy (see Table IV) was used as an input for the breath diagnostic network 24 autoencoder and softmax. Additional VOCs were added in order of decreasing accuracy. The size of the data used in this experiment is 504 sample (252 benign and 252 malignant), with 75% of the data used for training and 25% of the data for testing.

TABLE V
Analysis of combinations of VOCs as biomarkers for cancer.

| # of Fused VOC | Sensitivity | Specificity | Accuracy |
|---|---|---|---|
| 1 | 77.59 | 73.53 | 75.4 |
| 2 | 76.79 | 80 | 78.57 |
| 3 | 78.33 | 83.33 | 80.95 |
| 4 | 71.93 | 86.96 | 80.16 |
| 5 | 72.41 | 89.71 | 81.75 |
| 6 | 75 | 87.88 | 81.75 |
| 7 | 79.31 | 77.94 | 78.57 |
| 8 | 79.31 | 76.47 | 77.78 |
| 9 | 74.24 | 78.33 | 76.19 |
| 10 | 74.6 | 80.95 | 77.78 |
| 11 | 67.65 | 77.59 | 72.22 |
| 12 | 77.19 | 75.36 | 76.19 |
| 13 | 78.46 | 75.41 | 76.98 |
| 14 | 77.14 | 80.36 | 78.57 |
| 15 | 69.23 | 83.61 | 76.19 |
| 16 | 73.13 | 77.97 | 75.4 |
| 17 | 71.88 | 80.65 | 76.19 |
| 18 | 81.97 | 69.23 | 75.4 |
| 19 | 74.14 | 72.06 | 73.02 |
| 20 | 74.6 | 76.19 | 75.4 |
| 21 | 79.03 | 75 | 76.98 |
| 22 | 68.33 | 81.82 | 75.4 |
| 23 | 70 | 85.71 | 76.98 |
| 24 | 78.69 | 75.38 | 76.98 |
| 25 | 77.46 | 74.55 | 76.19 |
| 26 | 76.67 | 74.24 | 75.4 |
| 27 | 71.23 | 88.68 | 78.57 |

The classification accuracy using the VOCs ranges from about 72% to about 82%, (regardless the number of fused VOCs). In some embodiments, the breath indicators 18 entered as input into the breath diagnostic network 24 comprise all 27 VOCs identified in Table IV. In other embodiments, fewer of the VOCs may be used to distinguish between malignant and benign nodules.

Training the Shape Diagnostic Network and Appearance Diagnostic Network. The breath test data for 75 patients with lung cancer and 75 patients with benign pulmonary nodules was used to train and optimize the shape and appearance diagnostic networks 20, 22. The parametric data obtained from the CT images (volume/size, SHs based shape complexity, and appearance using the Gibbs energy) for these same patients is integrated into the CAD system 10, as shown in FIGS. 1 and 7A-D.

The algorithms described in U.S. Pat. No. 9,230,320 contemplate use of growth rate of lung nodules as a factor in diagnosis. However, determination of growth rate require multiple scans of a subject patient. Growth rate is replaced herein with nodule size to allow a diagnosis based on a single scan of a subject patient. A k-NN classifier receives nodule size (i.e., diameter) as an input and outputs a size-based initial classification 36 of the nodule as malignant or benign.

The disclosed computer aided diagnostic system may be embodied in computer program instructions stored on a non-transitory computer readable storage medium configured to be executed by a computing system. The computing system utilized in conjunction with the computer aided diagnostic system described herein will typically include a processor in communication with a memory, and a network interface. Power, ground, clock, and other signals and circuitry are not discussed, but will be generally understood and easily implemented by those ordinarily skilled in the art. The processor, in some embodiments, is at least one microcontroller or general purpose microprocessor that reads its program from memory. The memory, in some embodiments, includes one or more types such as solid-state memory, magnetic memory, optical memory, or other computer-readable, non-transient storage media. In certain embodiments, the memory includes instructions that, when executed by the processor, cause the computing system to perform a certain action. Computing system also preferably includes a network interface connecting the computing system to a data network for electronic communication of data between the computing system and other devices attached to the network. In certain embodiments, the processor includes one or more processors and the memory includes one or more memories. In some embodiments, computing system is defined by one or more physical computing devices as described above. In other embodiments, the computing system may be defined by a virtual system hosted on one or more physical computing devices as described above.

While this specification primarily describes a non-invasive computer-aided system and method for detection of lung cancer receiving and analyzing data from a single CT scan (appearance, shape, size) and VOCs in a single exhaled breath sample, it should be understood that the system is capable of receiving and analyzing data from additional, fewer, or different sources. For example, the disclosed system is capable of generating a diagnosis using the three CT scan classifiers without breath data, using two of the CT scan classifiers with breath data, or with other classifiers not discussed herein, such as nodule growth rate or other biomarkers for cancer. It should also be understood that this system is capable of generating a diagnosis for cancers originating in locations other than the lung by changing the source data, e.g., replacing the panel of VOC biomarkers for lung cancer with a panel of VOC biomarkers for ovarian cancer or breast cancer.

Various aspects of different embodiments of the present invention are expressed in paragraphs X1 and X2 as follows:

X1. One aspect of the present invention pertains to a computer-aided method for identifying the presence or absence of a cancer disease state, the method comprising receiving a plurality of measurable indicators of the presence or absence of a cancer disease state in a subject; generating an initial classification probability, using a neural network, from each of the plurality of measurable indicators; assigning a weight to each initial classification probability using the neural network; and generating a final classification by integrating the initial classification probabilities based on their respective weights using the neural network; wherein the final classification is designating the presence or absence of a cancer disease state in the subject.

X2. Another aspect of the present invention pertains to a non-transitory computer readable storage medium having computer program instructions stored thereon that, when executed by a processor, cause the processor to perform the following instructions: receiving a plurality measurable of indicators of the presence or absence of a cancer disease state in a subject; generating an initial classification probability, using a neural network, from each of the plurality of measurable indicators; assigning a weight to each initial classification probability using the neural network; and generating a final classification by integrating the initial classification probabilities based on their respective weights using the neural network; wherein the final classification is designating the presence or absence of a cancer disease state in the subject.

Other embodiments pertain to any of the previous statements X1 or X2 which are combined with one or more of the following other aspects.

Wherein the neural network includes a first stage and a second stage, each stage including a dimensionality reducer and a softmax layer.

Wherein the dimensionality reducer is an autoencoder.

Wherein the autoencoder of the first stage includes a greater number of hidden layers than the autoencoder of the second stage.

Wherein generating the initial classification probability is enacted by the first stage, and wherein assigning the weight and generating the final classification is enacted by the second stage.

Wherein the plurality of measurable indicators includes at least two of a spherical harmonic shape analysis of an anatomical structure of the subject, a size of the anatomical structure, a quantified appearance of the anatomical structure, and subject values of volatile organic compounds in an exhaled breath sample from the subject.

Wherein the plurality of measurable indicators includes at least three of a spherical harmonic shape analysis of an anatomical structure of the subject, a size of the anatomical structure, a quantified appearance of the anatomical structure, and subject values of volatile organic compounds in an exhaled breath sample from the subject.

Wherein the plurality of measurable indicators includes a spherical harmonic shape analysis of an anatomical structure of the subject, a size of the anatomical structure, a quantified appearance of the anatomical structure, and subject values of volatile organic compounds in an exhaled breath sample from the subject.

Further comprising obtaining an additional measurable indicator of the presence or absence of a cancer disease state in the subject; and assigning a weight to the additional measurable indicator using the neural network; and wherein generating the final classification includes generating the final classification by integrating the initial classification probabilities and the additional measurable indicator based on their respective weights using the neural network.

Wherein the plurality of measurable indicators includes a spherical harmonic shape analysis of an anatomical structure of the subject, a quantified appearance of the anatomical structure, and subject values of volatile organic compounds in an exhaled breath sample from the subject; and wherein the additional measurable indicator includes a size of the anatomical structure.

The foregoing detailed description is given primarily for clearness of understanding and no unnecessary limitations are to be understood therefrom for modifications can be made by those skilled in the art upon reading this disclosure and may be made without departing from the spirit of the invention. While this invention is discussed primarily in connection with the detection of lung cancer, it should be understood that cancers not primary to the lung or cancers that have not metastasized to the lung may also be detected. Furthermore, it should be understood the different biomarkers or other data may be used to generate initial classification probabilities.

What is claimed is:

1) A computer-aided method for identifying the presence or absence of a cancer disease state, the method comprising:
receiving a plurality of measurable indicators of the presence or absence of a cancer disease state in a subject;
generating an initial classification probability, using a neural network, from each of the plurality of measurable indicators;
assigning a weight to each initial classification probability using the neural network; and
generating a final classification by integrating the initial classification probabilities based on their respective weights using the neural network;
wherein the final classification is designating the presence or absence of a cancer disease state in the subject.

2) The method of claim 1, wherein the neural network includes a first stage and a second stage, each stage including a dimensionality reducer and a softmax layer.

3) The method of claim 2, wherein the dimensionality reducer is an autoencoder.

4) The method of claim 2, wherein generating the initial classification probability is enacted by the first stage, and wherein assigning the weight and generating the final classification is enacted by the second stage.

5) The method of claim 1, wherein the plurality of measurable indicators includes at least two of a spherical harmonic shape analysis of an anatomical structure of the subject, a size of the anatomical structure, a quantified appearance of the anatomical structure, and subject values of volatile organic compounds in an exhaled breath sample from the subject.

6) The method of claim 1, wherein the plurality of measurable indicators includes at least three of a spherical harmonic shape analysis of an anatomical structure of the subject, a size of the anatomical structure, a quantified appearance of the anatomical structure, and subject values of volatile organic compounds in an exhaled breath sample from the subject.

7) The method of claim 1, wherein the plurality of measurable indicators includes a spherical harmonic shape analysis of an anatomical structure of the subject, a size of the anatomical structure, a quantified appearance of the anatomical structure, and subject values of volatile organic compounds in an exhaled breath sample from the subject.

8) The method of claim 1, further comprising:
obtaining an additional measurable indicator of the presence or absence of a cancer disease state in the subject; and
assigning a weight to the additional measurable indicator using the neural network; and
wherein generating the final classification includes generating the final classification by integrating the initial classification probabilities and the additional measurable indicator based on their respective weights using the neural network.

9) The method of claim 8, wherein the plurality of measurable indicators includes a spherical harmonic shape analysis of an anatomical structure of the subject, a quantified appearance of the anatomical structure, and subject values of volatile organic compounds in an exhaled breath sample from the subject; and
wherein the additional measurable indicator includes a size of the anatomical structure.

10) A non-transitory computer readable storage medium having computer program instructions stored thereon that, when executed by a processor, cause the processor to perform the following instructions:
receiving a plurality of measurable indicators of the presence or absence of a cancer disease state in a subject;
generating an initial classification probability, using a neural network, from each of the plurality of measurable indicators;
assigning a weight to each initial classification probability using the neural network; and
generating a final classification by integrating the initial classification probabilities based on their respective weights using the neural network;
wherein the final classification is designating the presence or absence of a cancer disease state in the subject.

11) The non-transitory computer readable storage medium of claim 10, wherein the neural network includes a first stage and a second stage, each stage including a dimensionality reducer and a softmax layer.

12) The non-transitory computer readable storage medium of claim 11, wherein the dimensionality reducer is an autoencoder.

13) The non-transitory computer readable storage medium of claim 11, wherein generating the initial classification probability is enacted by the first stage, and wherein assigning the weight and generating the final classification is enacted by the second stage.

14) The non-transitory computer readable storage medium of claim 10, wherein the plurality of measurable indicators includes at least two of a spherical harmonic shape analysis of an anatomical structure of the subject, a size of the anatomical structure, a quantified appearance of the anatomical structure, and subject values of volatile organic compounds in an exhaled breath sample from the subject.

15) The non-transitory computer readable storage medium of claim 10, wherein the plurality of measurable indicators includes at least three of a spherical harmonic shape analysis of an anatomical structure of the subject, a size of the anatomical structure, a quantified appearance of the anatomical structure, and subject values of volatile organic compounds in an exhaled breath sample from the subject.

16) The non-transitory computer readable storage medium of claim 10, wherein the plurality of measurable indicators includes a spherical harmonic shape analysis of an anatomical structure of the subject, a size of the anatomical structure, a quantified appearance of the anatomical structure, and subject values of volatile organic compounds in an exhaled breath sample from the subject.

17) The non-transitory computer readable storage medium of claim 10, further comprising:
obtaining an additional measurable indicator of the presence or absence of a cancer disease state in the subject; and
assigning a weight to the additional measurable indicator using the neural network; and
wherein generating the final classification includes generating the final classification by integrating the initial classification probabilities and the additional measurable indicator based on their respective weights using the neural network.
18) The non-transitory computer readable storage medium of claim 17, wherein the plurality of measurable indicators includes a spherical harmonic shape analysis of an anatomical structure of the subject, a quantified appearance of the anatomical structure, and subject values of volatile organic compounds in an exhaled breath sample from the subject; and
wherein the additional measurable indicator includes a size of the anatomical structure.
